(19) United States
(12) Patent Application Publication
(10) Pub. No.: US 2021/0121649 A1
(43) Pub. Date: Apr. 29, 2021

(54) PORTABLE REBREATHING SYSTEM WITH STAGED ADDITION OF OXYGEN ENRICHMENT

(71) Applicant: MIROLA IP AB, Örebro (SE)

(72) Inventors: Ola TELBY, Karlstad (SE); Jonny BERZELIUS, Stockholm (SE)

(73) Assignee: MIROLA IP AB, Örebro (SE)

(21) Appl. No.: 16/765,302

(22) PCT Filed: May 27, 2019
(86) PCT No.: PCT/EP2019/063662

§ 371 Date: May 19, 2020

(30) Foreign Application Priority Data
Jul. 17, 2018 (SE) 1830221-6

(51) Int. Cl. A61M 16/00; A61M 16/06; A61M 16/20; A61M 16/08

(52) U.S. Cl. CPC A61M 16/0045; A61M 16/06; A61M 16/208; A61M 2205/3334; A61M 16/0875; A61M 16/0078; A61M 2202/0208; A61M 16/206

(57) ABSTRACT

The invention is related to a portable rebreathing system for closed rebreathing. In order to minimize consumption of oxygen during rebreathing mode while safeguarding correct oxygen concentration, oxygen is added into the breathing passage using staged addition of oxygen via at least three individual oxygen supply valves 51, 52, 53. The two first oxygen supply valves are calibrated nozzles where one nozzle 51 is constantly delivering a predetermined amount of oxygen during normal breathing and the second nozzle 52 adds more oxygen at a second predetermined amount when the person to be treated is breathing heavily. The third valve is only opened manually and delivers a short burst of oxygen that fills the rebreathing system and its counter lung within seconds.

### FIELD OF THE INVENTION

The present invention relates to a portable rebreathing system with pressurized oxygen enrichment, said portable rebreathing system comprising a breathing mask, a carbon dioxide scrubber, a counter lung and an oxygen supply port connected via a hose to a pressurized oxygen source.

### BACKGROUND INFORMATION

The surrounding air consists of about 21% of oxygen. At each inhalation, the body extracts about 5% units of that oxygen and the remaining 16% of oxygen is exhaled to the atmosphere again together with C0₂ which is about 5% of the volume exhaled. To reduce the amount of oxygen gas needed in a breathing equipment, and make it possible to reuse the oxygen exhaled, closed circuit breathing apparatus also called rebreathers are used. In a rebreather, the produced C0₂ is absorbed in a scrubber material, most often calcium hydroxide or soda lime. Rebreathers can also be used to provide high oxygen fractions for medical purposes without wasting a lot of oxygen.

Several prior art systems provide closed re-breathing systems to be used in oxygen depleted or toxic environment. In those system is most often used a carbon dioxide scrubber for the exhalation flow that allows the exhaled air flow to be used again during inhalation. This type of rescue breathing system is typically used for miners or people caught in other areas with toxic fumes.

Some of this type of rescue breathing systems also include non pressurised oxygen generators that may be activated chemically by mixing chemicals or using a special ignitable oxygen producing candles. With oxygen generators, the operating time for the rescue breathing systems could be extended and a small volume of oxygen is added into the rebreathing circuit keeping the total breathing volume constant.

Examples of these re-breathing systems could be seen in;
GB2189152, Emergency escape breathing apparatus, with one-way valves in a breathing mask, using a counter lung connected to an O₂ tank covering the entire head and a CO₂ scrubbing filter.
GB2233905; Emergency escape breathing apparatus, with one-way valves in a breathing mask, using a counter lung covering the entire head and a filter capable of both CO₂ scrubbing and O₂ generation.
U.S. Pat. No. 5,113,854, Protective hood with CO₂ scrubbing and a cylinder supplying oxygen into the hood.
US2011/0277768, Protective hood with valves preventing inhalation via scrubber and a cylinder supplying oxygen into the hood.

Still a number of rebreathing systems have been proposed such as
U.S. Pat. No. 4,205,673 (1980), with an ignitable oxygen producing candle;
U.S. Pat. No. 4,172,454 (1979), with a complete protection suit;
U.S. Pat. No. 4,246,229 (1981), with a chemical oxygen generator;
U.S. Pat. No. 4,817,597 (1989), with heat dissipating channel over the counter lung;
U.S. Pat. No. 5,267,558 (1993), Chemical oxygen generator with flow distributor through scrubber;
US2014/0014098; with visible indicator for oxygen shortage

Re-breathing systems have also been proposed for controlled treatment of persons with reduced lung capacity, or otherwise show low oxygen saturation in the blood. In such cases is also an increased oxygen content in the inhaled flow sought for, sometimes raised from the normal 21% O₂ content in ambient air and up to 100% O₂ content.

Rescue vehicles are often equipped with large oxygen tanks that may supply pure oxygen into breathing masks or into nozzles applied into the nostrils. The problem is that the oxygen is consumed rapidly and most of it is wasted during exhalation. Another problem is the total weight of the system which cause strains on the rescue personnel and may prevent quick appliance to patients in real field situations. Conventionally, the oxygen has been supplied from a large pressurized oxygen cylinder, in loaded state pressurized to 200-300 bars, directly to a breathing mask covering the mouth and nose, or via nozzles entered directly into the nostrils. However, a huge part of the oxygen supplied has been wasted.

Most of the rebreathing systems developed for rescue purposes in oxygen depleted environment could not be used for intensified oxygen treatment, so rescue personnel need to bring along bulky and heavy oxygen tanks that conventionally could only be connected to one person at the time.

The need for many small rebreathing systems to be used for intensified oxygen treatment became evident in Sweden after a large fire in a discotheque, where almost a hundred youngsters were rescued but with smoke affected lungs. Even if a tenfold of ambulances arrived at the accident scene, only a tenfold of persons were given the aid of increased oxygen treatment. This since each rescue vehicle only had one bulky oxygen tank and one connector with a single mouth piece.

WO2014/035330 discloses a rebreathing system used for extending supply of oxygen to the rebreathing circuit. As disclosed in WO2014/035330 is the necessity and use of this rebreathing system in detail described. In this rebreathing system is a single two-way valve used to shut off a breathing passage when the pressure of the external oxygen source drops.

SE1730011-2 discloses a further development of WO2014/035330 with improved functionality that minimizes the dead volume of exhaled CO₂ rich air that may be inhaled in subsequent inhalation. Once the exhalation flow has passed one valve in a three-valve seating close to the mouthpiece, the CO₂-rich air could not be inhaled again until this exhaled volume has passed through the carbon dioxide scrubber.

### SUMMARY OF THE INVENTION

The present invention is a further development of rebreathers making them more reliable as to delivery of the target oxygen enrichment while extending the operational time for one rebreather connected to an oxygen source. Further, the rebreather must be easy to apply and activate, and intuitively activated such that longest possible treatment time may be obtained when using the oxygen available.

The invention is a portable rebreathing system for closed rebreathing, comprising
a breathing mask,
a common valve housing connected with a mask connector to the breathing mask;
a carbon dioxide scrubber connected with a scrubber connector to the common valve housing;
a counter lung connected with a counter lung connector to the carbon dioxide scrubber;
an oxygen supply port and at least one ambient air port arranged in the common valve housing;
a pressurized oxygen source connected to the oxygen supply port via a hose.

According to the invention, the oxygen supply port is in communication with at least three oxygen supply valves and all oxygen supply valves have outlets emanating into an inhale flow passage in the common valve housing. The first oxygen supply valve is a constant flow rate nozzle valve delivering oxygen through a small restriction at a first flow rate when the pressurized oxygen source is connected. The second oxygen supply valve is a constant flow rate nozzle valve delivering oxygen through a small restriction at a second flow rate equal to or exceeding the first flow rate when inhalation is excessive. The third oxygen supply valve is a nozzle valve delivering oxygen through a restriction at a third flow rate exceeding the first flow rate by at least 40 times when a manual activation knob in the common valve housing is pushed down.

This general design of the rebreathing system with staged addition of oxygen in three distinct stages by individual nozzles will establish a low but sufficient consumption of oxygen during established rebreathing during normal breathing frequency, and automatic enrichment if the person to be treated breathe more heavily due to medical reasons or physical work. A third distinctive addition at much larger rate, activated by pushing in a knob manually, allows the rescue personnel to quickly fill the rebreather with oxygen in order to set up the rebreathing system at start, as well as allowing the person to be treated to increase oxygen temporarily.

According to a preferred embodiment, also the oxygen supply port is in communication with a shut-off valve in the common valve housing closing an alternative breathing passage to an ambient port when oxygen pressure is applied in the oxygen supply port and opening an alternative breathing passage connected to an ambient air port when no oxygen pressure is applied in the oxygen supply port. This enables the rescue personnel to apply the breathing mask onto the face of the person to be treated before oxygen supply is activated, while allowing the person to be treated to continue breathing via the alternative breathing passage until the very moment when oxygen is turned on.

Further, according to yet a preferred embodiment, a flexible membrane is arranged as a wall in the inhalation flow passage allowing deflection into the inhalation flow passage when a flow rate in the inhalation flow passage exceeds a predetermined level. The deflecting membrane may be used to activate the second oxygen supply valve depending on increased breathing which automatically lowers the pressure on the membrane. The second stage of oxygen addition may thus be activated as a consequence to excessive breathing.

In yet a preferred embodiment the common valve housing has a cylindrical form and that the membrane is a cylindrical flexible disc with its periphery arranged fixed and sealed to the inside of the cylindrical common valve housing with one side of the membrane exposed to the inhalation flow passage in a narrow flow path that locally increases speed of flow and thus creates a lower pressure on the exposed side of the membrane.

The flexible membrane may also deflect a pivot lever when the flow rate in the inhalation flow passage exceeds the predetermined level and said deflection of the pivot lever opens the second oxygen supply valve. Such a pivot lever may be used to increase the opening movement on the second oxygen supply valve compared with a smaller deflection movement of the membrane, if the lever length is smaller for the membrane than the lever length for the valve located on the other side of the pivot point of the pivot lever.

In another preferred embodiment, the flexible membrane is also deflectable by a manual activation knob which knob when depressed fully deflects the pivot lever further such that the additional deflection of the pivot lever opens also the third oxygen supply valve. This simplifies the valve regulation design as the same membrane movement and lever activates the 2 additional valves in sequence, and no special manual activator needs to be included.

In a preferred embodiment is the first oxygen supply valve a constant flow rate nozzle valve, with a calibrated bore through the nozzle delivering a constant flow at a rate of 0.5-1.5 liter of oxygen per minute. These constant flow rate nozzles are readily available on the market at low cost but made with small variations between individual nozzle with same nominal capacity.

Hence, the second oxygen supply valve may also be constant flow rate nozzle valve, with a calibrated bore through the nozzle delivering a constant flow at a rate of 1.0-2.0 liter of oxygen per minute.

In a further embodiment may the third oxygen supply valve be a restriction which when opened delivers a constant flow at a rate of 10-100 liter of oxygen per minute. The third oxygen supply valve preferably delivers a constant flow at a rate of 50-70 liter of oxygen per minute, and capable of filling the system and an expanded counter lung in 3 seconds. A short burst of oxygen may thus fill the entire rebreathing system, making it possible to start the rebreathing at high oxygen concentration.

### BRIEF DESCRIPTION OF THE DRAWINGS

The foregoing aspects and advantages of this invention will become more readily appreciated as the same become better understood by reference to the following detailed description, when taken in conjunction with the accompanying schematically drawings, wherein:

FIG. 1a shows a side view in a cross section of a first schematic embodiment of the rebreathing system according to the invention, here during an inhalation phase;

FIG. 1b shows same side view as in FIG. 1a but here during an exhalation phase;

FIG. 2a shows a flat view as well as a side view in a cross section of a valve seat member used in one embodiment of the invention;

FIG. 2b shows same views as in FIG. 2a but with valve members attached and breathing directly to atmosphere;

FIG. 2c shows same views as in FIG. 2b but in rebreathing mode during an exhalation phase;

FIG. 2d shows same views as in FIG. 2b but in rebreathing mode during an inhalation phase;

FIG. 3a shows a side view in a cross section of a first schematic embodiment of the common valve housing during normal breathing;

FIG. 3b shows the same view as in FIG. 3a but during excessive breathing;

FIG. 3c shows the same view as in FIG. 3b but during maximum activation of a manual activation knob;

FIG. 3d shows the same view as in FIGS. 3a-3c but with no oxygen pressure applied when breathing takes place directly to atmosphere;

FIG. 3e show an example of a constant flow rate nozzle valve;

FIG. 4a-4c shows the alternative breathing passage used in FIG. 3d with no oxygen pressure applied;

FIG. 5 shows a complete prototype of an embodiment of the invention.

However, it should be stressed that the drawings only visualize the concepts of the invention, as presentable in 2 dimensional drawings. Some channels may for instance utilize the option to be routed not only in the 2 dimensions shown, but also may be routed in 3 dimensions fully utilizing the total volume of the common valve housing. The pressurized oxygen source may be a bottle or an oxygen outlet in a hospital.

### DETAILED DESCRIPTION OF THE ENABLING EMBODIMENTS

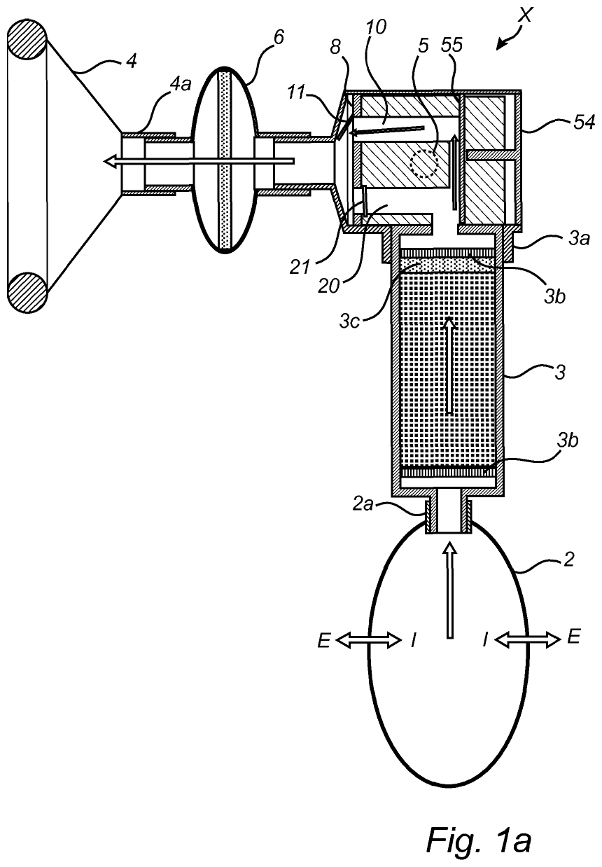

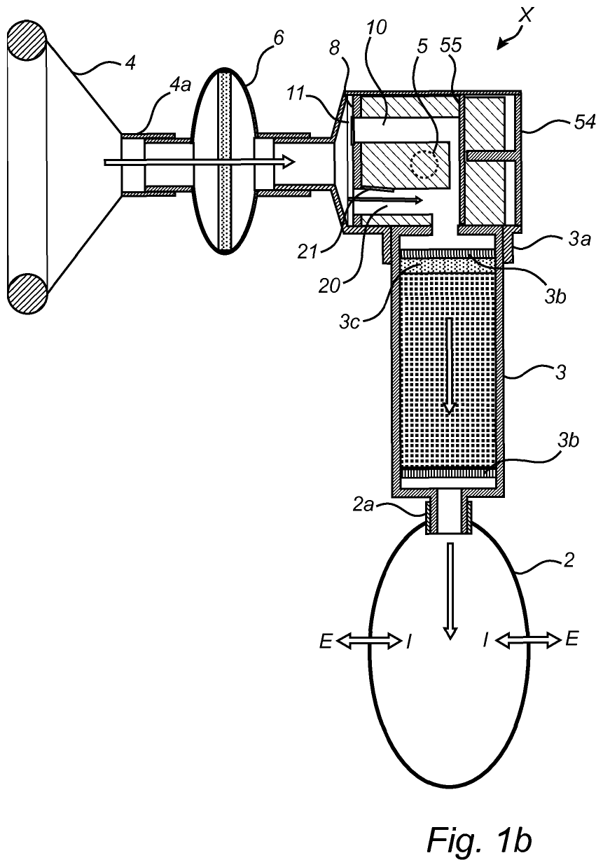

In FIG. 1a, a side view in a cross section of a first schematic embodiment of the rebreathing system according to the invention is shown, here during an inhalation phase. The inhalation flow through the rebreather is shown with arrows having a double flow line.

The rebreather has a breathing mask 4 that is to be applied over the mouth and nose of a person to be treated, said mask typically made in flexible rubber material like silicone rubber.

The breathing mask 4 is in turn connected to a bio-filter 6 with a mask connector 4a gripping over a congruent circular connector of the bio-filter with a press fit. The bio-filter is connected to the common valve housing X with a similar connection. The bio-filter is used to avoid ingress of biological material, like vomit from a person to be treated as well as bacteria. After usage may the bio-filter be exchanged and the non-contaminated rebreathing kit may be used for another person, not needing sterilization of the common valve housing.

The common valve housing X has an inhalation flow passage 10 and an exhalation flow passage 20. If the inhalation phase is to start in FIG. 1a is a counter lung 2 inflated, and during the inhalation phase, breathing air is drawn from the counter lung 2 through a carbon dioxide scrubber 3 and further on passing over a membrane 55 in the common valve housing X. The inhalation flow is thereafter diverted 90 degrees into a channel 10 and passing a first one-way check valve 11. The check valve 11 is typically made in rubber and may have any suitable form as a rhomboid or circular form. The counter lung 2 is simply a flexible bag in polymeric material and is attached with a counter lung connector 2a to the carbon dioxide scrubber in the same manner as the connector 4a for the breathing mask. The counter lung 2 expands in the direction E during the exhalation and retracts in the direction I during inhalation. The carbon dioxide scrubber is filled with any active material that binds CO₂, typically in powder form, with diffusors 3b in both ends. The upper end of the carbon dioxide scrubber is also equipped with a fine mesh filter 3c that prevents scrubber material from entering the common valve housing.

The common valve housing X is also equipped with an oxygen supply port 5, and a manual activation knob 54, which will be more described later.

In FIG. 1b a side view is shown in a cross section of the first schematic embodiment of the rebreathing system according to the invention, here during an exhalation phase. The exhalation flow through the rebreather is shown with arrows having a double flow line. In contrast to the flow pattern shown in FIG. 1 is the exhalation flow opening a second one-way check valve 21 into an exhalation flow passage 20, while the increase pressure during exhalation closes the first one-way check valve 11. The exhalation flow is diverted through the carbon dioxide scrubber 3 and finally to the counter lung 2.

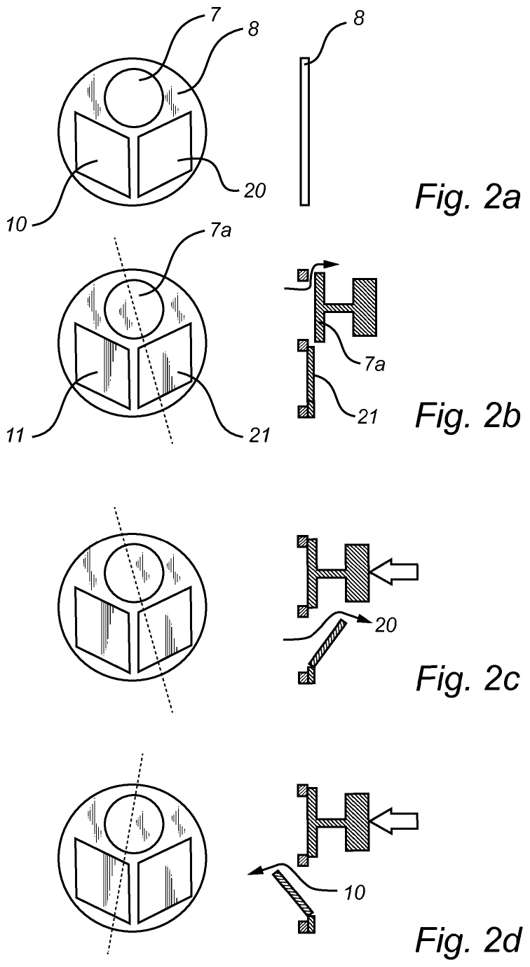

In FIGS. 2a to 2d the valve seat member 8 and associated valves during different phases of breathing are shown schematically. FIG. 2a shows a flat view as well as a side view in a cross section of the valve seat member 8 alone. The valve seat member has a first opening for an alternative breathing passage 7 open when no oxygen addition is activated and an opening for the inhalation flow passage 10 as well as an opening for the exhalation flow passage 20. In this embodiment the inhalation and exhalation passages have a rhomboid form enabling the largest flow area in these passages when the common valve housing has a tubular form, but these passages may equally well be circular. FIG. 2b shows same views as in FIG. 2a but with valve members attached and breathing directly to atmosphere in an alternative breathing passage 7. A shut off valve 7a is open as long as no oxygen pressure is connected and the one-way check valve 21 is closed as no pressure could build up on the valve 21. FIG. 2c, shows same views as in FIG. 2b but in rebreathing mode during an exhalation phase. When rebreathing is to be activated is simply oxygen pressure applied on the shut-off valve (as indicated with the grey arrow), and then the pressure builds up on the one-way check valve 21 and will open it to the exhalation flow passage. FIG. 2d, shows same views as in FIG. 2b but in rebreathing mode during an inhalation phase, and then the pressure drops on the one-way check valve 11 and will open the inhalation flow passage.

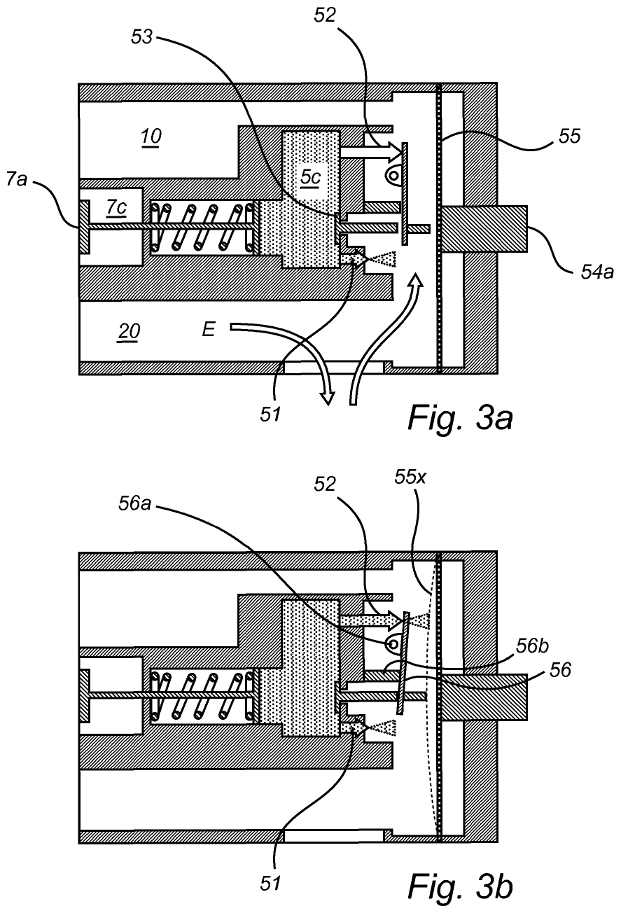

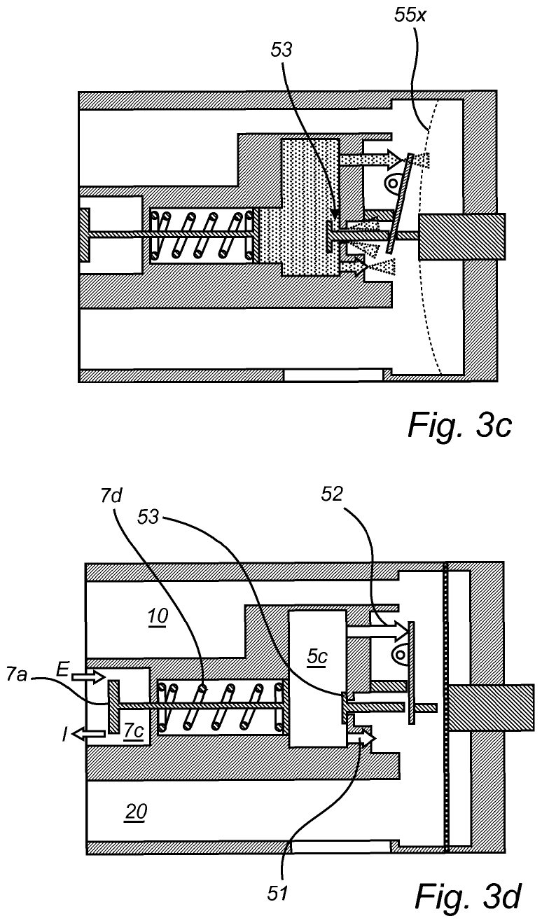

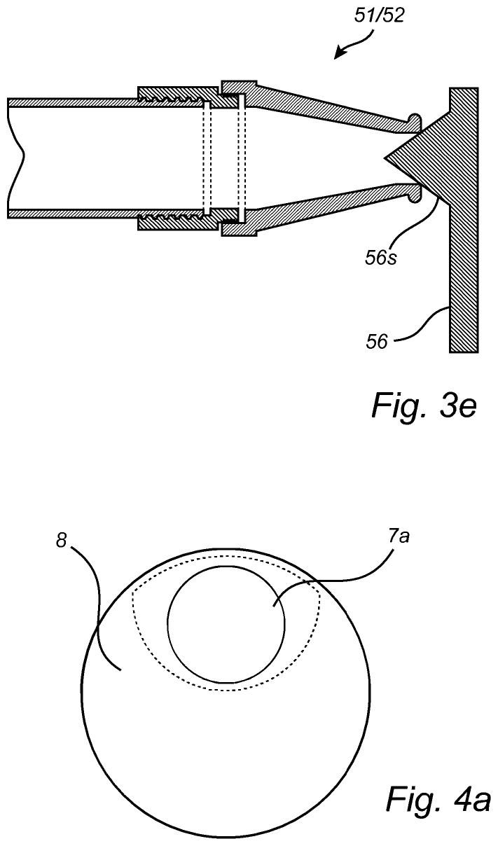

The functionality of the common valve housing X will be described more in detail with reference to FIGS. 3a to 3d. In order to simplify, the schematic cross section is made through the inhalation and exhalation flow passages 10 and 20, even though they may be located in the clock positions 4 and 8 as shown in FIG. 2a.

FIG. 3a shows a side view in this schematic cross section of a first schematic embodiment of the common valve housing during activated rebreathing with addition of oxygen. A pressure chamber 5c is pressurized with oxygen at any selected pressure added via an oxygen supply port 5 in the common valve housing X. Typically, the pressure in the pressure chamber is regulated to a level of 4 bar, using any standard pressure regulator between the oxygen source and the common valve housing X. This pressure chamber is in direct communication with;
a first oxygen supply valve 51,
a second oxygen supply valve 52,
a third oxygen supply valve 53, and
a piston connected to a spring biased shut-off valve 7a.

During normal breathing, only the first oxygen supply valve 51 is open as indicated in FIG. 3a. This first oxygen supply valve delivers a constant flow of oxygen at a constant flow rate of about 0.5-1.5 liter of oxygen per minute when the connection to the oxygen source has been made. Typically, 1 liter of oxygen per minute is fully sufficient for replacing the amount of CO₂ in the exhaled air for an adult person when breathing normally. The first oxygen supply valve 51 is a constant flow rate nozzle valve with a calibrated bore that are available as standard nozzles and could be replaced if needed. However, this calibrated nozzle safeguards the efficient use of available oxygen for maximum length of usage and minimum consumption.

FIG. 3b shows the same view as in FIG. 3a but during excessive breathing. In this state, the person treated is most often hyperventilating. The flow of inhalation air increases and that causes a pressure drop over the flexible membrane 55 that deflects to a position 55x as indicated in FIG. 3b. The passage over the membrane may preferably be designed as a narrow throat that increase speed of passing air and this increase the pressure drop. During this deflection, the flexible membrane 55 is pushing a pivot lever 56 around a pivot point 56a and against a pivot spring 56b. When the deflection pushes the pivot lever 56, the second oxygen supply valve 52 is also opened. This second oxygen supply valve delivers a constant flow of oxygen at a constant flow rate of about 1-2 liter of oxygen per minute when the connection to the oxygen source has been made. Typically, an additional 1 liter of oxygen per minute is fully sufficient for replacing the amount of CO₂ in the exhaled air for an adult person when hyperventilating. The second oxygen supply valve 52 is also a constant flow rate nozzle valve with a calibrated bore that are available as standard nozzles and could be replaced if needed. However, this calibrated nozzle safeguards the efficient use of available oxygen for maximum length of usage and minimum consumption and is only open during hyperventilation.

FIG. 3c shows the same view as in FIG. 3b but during maximum activation of a manual activation knob 54. Here, only the stem 54a is shown on the activation knob shown in FIG. 1a. This state is only manually activated when the rebreather is to be started and pushing the activator knob to the bottom could fill the counter lung in a couple of seconds. This will set the starting conditions for the rebreather and the person to be treated will be fed by pure oxygen for maximum assistance and all CO₂exhaled will be caught in the carbon dioxide scrubber. When the knob is pressed to the bottom, the additional deflection of the flexible membrane 55 is pushing the pivot lever 56 further around a pivot point 56a and against a pivot spring 56b. When the additional deflection pushes the pivot lever 56, the third oxygen supply valve 53 is also opened. This third oxygen supply valve delivers a constant flow of oxygen at a constant flow rate of about 10-100 liter, preferably 50-70 liter of oxygen per minute when the connection to the oxygen source has been made. The third oxygen supply valve 53 may be a simpler non-calibrated valve with a restriction gap capable of filling the system and an expanded counter lung in 1-3 seconds.

FIG. 3d shows the same view as in FIGS. 3a-3c but with no oxygen pressure applied when breathing takes place directly to atmosphere. As no pressure is established in the pressure chamber 5c are all oxygen supply valves idle. The shut-off valve 7a is opened by a return spring member allowing establishment of an alternative breathing passage to the ambient air chamber 7c.

FIG. 3e show an example of a constant flow rate nozzle valve that may be used as the first oxygen supply valve 51 and/or as the second oxygen supply valve 52. Here is also shown the pivot lever 56 (not used with nozzle 51) that may close the nozzle and may also have a sealing member 56s attached to the pivot lever. The nozzles are easily exchanged as they are mounted by threads and are manufactured in large series with calibrated flow capacity for any specific supply pressure.

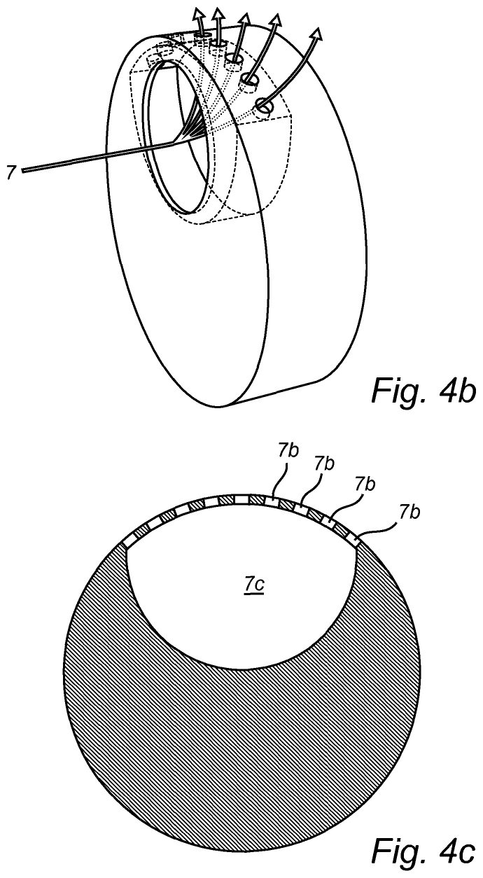

FIGS. 4a-4c show the alternative breathing passage used in FIG. 3d with no oxygen pressure applied. A flat view of the valve seat member 8 is shown in FIG. 4a with the shut-off valve 7a and the contour of the ambient air chamber 7c shown in phantom lines. FIG. 4b shows the alternative breathing passage 7 through the ambient air chamber, which finally ends in a multiple of outlets 7b as shown in FIG. 4c.

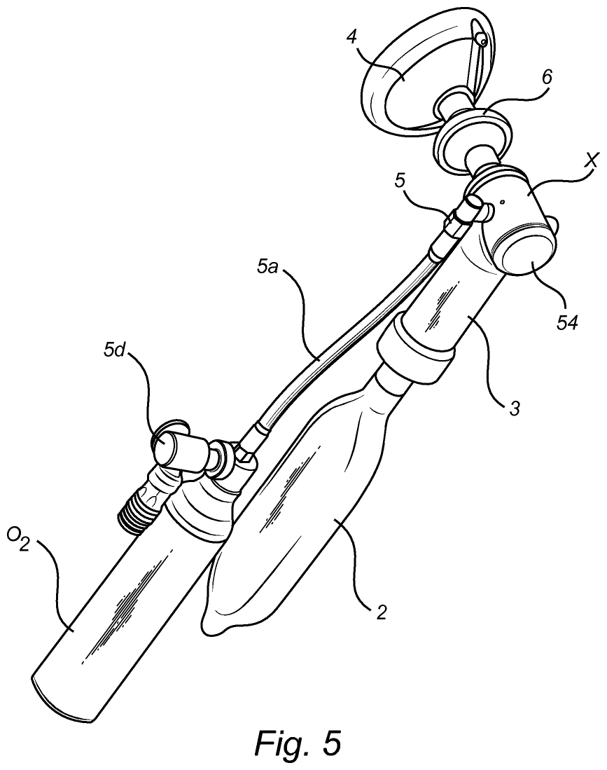

Finally, a complete prototype of an embodiment of the invention is shown in FIG. 5. The rebreathing unit is here shown connected to an oxygen source O₂ in form of a small pressure bottle. A standard pressure regulator 5d connects to the common valve housing X via a pressure hose 5a. The small tubular common valve housing X contains all the necessary valves, with a tubular carbon dioxide scrubber 3 connected orthogonally to the common valve housing. The tubular form is chosen to allow simple and steady handling of the rebreather with one hand.

1. A portable rebreathing system for closed rebreathing, said portable rebreathing system comprising
a breathing mask,
a common valve housing connected with a mask connector to the breathing mask;
a carbon dioxide scrubber connected with a scrubber connector to the common valve housing;
a counter lung connected with a counter lung connector to the carbon dioxide scrubber;
an oxygen supply port and at least one ambient air port arranged in the common valve housing;
a pressurized oxygen source connected to the oxygen supply port via a hose;
wherein the oxygen supply port is in communication with at least three oxygen supply valves, and all oxygen supply valves have outlets emanating into an inhale flow passage in the common valve housing; and
the first oxygen supply valve is a constant flow rate nozzle valve delivering oxygen through a small restriction at a first flow rate when the pressurized oxygen source is connected, and
the second oxygen supply valve is a constant flow rate nozzle valve delivering oxygen through a small restriction at a second flow rate equal to or exceeding the first flow rate when inhalation is excessive, and
the third oxygen supply valve is a nozzle valve delivering oxygen through a restriction at a third flow rate exceeding the first flow rate by at least 40 times when a manual activation knob in the common valve housing is pushed down.

2. A portable rebreathing system according to claim 1, wherein the oxygen supply port is in communication with a shut-off valve in the common valve housing closing an alternative breathing passage to the ambient port when oxygen pressure is applied in the oxygen supply port and opening an alternative breathing passage connected to an ambient air port when no oxygen pressure is applied in the oxygen supply port.

3. A portable rebreathing system according to claim 1, wherein a flexible membrane is arranged as a wall in the inhalation flow passage allowing deflection into the inhalation flow passage when a flow rate in the inhalation flow passage exceeds a predetermined level.

4. A portable rebreathing system according to claim 3, wherein the common valve housing has a cylindrical form and that the membrane is a cylindrical flexible disc with its periphery arranged fixed and sealed to the inside of the cylindrical common valve housing with one side of the membrane exposed to the inhalation flow passage in a narrow flow path that locally increases speed of flow and thus creates a lower pressure on the exposed side of the membrane.

5. A portable rebreathing system according to claim 3, wherein the flexible membrane deflects a pivot lever when the flow rate in the inhalation flow passage exceeds the predetermined level and said deflection of the pivot lever opens the second oxygen supply valve.

6. A portable rebreathing system according to claim 5, wherein the flexible membrane is also deflectable by a manual activation knob which knob when depressed fully deflects the pivot lever further such that the additional deflection of the pivot lever opens also the third oxygen supply valve.

7. A portable rebreathing system according to claim 1, wherein the first oxygen supply valve is a constant flow rate nozzle valve, with a calibrated bore through the nozzle delivering a constant flow at a rate of 0.5-1.5 liter of oxygen per minute.

8. A portable rebreathing system according to claim 1, wherein the second oxygen supply valve is a constant flow rate nozzle valve, with a calibrated bore through the nozzle delivering a constant flow at a rate of 1.0-2.0 liter of oxygen per minute.

9. A portable rebreathing system according to claim 1, wherein the third oxygen supply valve is a restriction which when opened delivers a constant flow at a rate of 10-100 liter of oxygen per minute.

10. A portable rebreathing system according to claim 9, wherein the third oxygen supply valve delivers a constant flow at a rate of 50-70 liter of oxygen per minute, and capable of filling the system and an expanded counter lung in 3 seconds.
